(19) United States
(12) Patent Application Publication
(10) Pub. No.: US 2007/0025525 A1
(43) Pub. Date: Feb. 1, 2007

(54) Means for improving patient positioning during X-ray imaging

(76) Inventor: Chaim Gilath, Rehovot (IL)

Correspondence Address: Angenehm Law Firm. Ltd., P.O. Box 48755, Coon Rapids, MN, 55448-0755, US

(21) Appl. No.: 11/179,402

(22) Filed: Jul. 12, 2005

(51) Int. Cl. A61B 6/08

(52) U.S. Cl. 378/206

(57) ABSTRACT

The present invention provides unique means and methods for improving patient positioning in relation to automatic exposure control (AEC) sensors before imaging takes place in an x-ray imaging system. The means for improving patient positioning comprises means adapted to project position and field size covered by the AEC system's sensors onto the patient in an X-ray imaging system provided with an x-ray tube, collimator provided with a light source, AEC system's sensors and an imaging device.

### FIELD OF THE INVENTION

The present invention relates to patient positioning in X-Ray imaging. More particularly, the present invention relates to means and methods for improving patient positioning relative to the automatic exposure control (AEC) sensor(s) before the imaging procedure is performed, by projecting the position and field size of the AEC sensors onto the patient.

### BACKGROUND OF THE INVENTION

Exposure of patients to radiation in X-ray radiography, fluoroscopy or angiography is typically controlled by an automatic exposure control (AEC) system based on one or more sensors such as for example ionization chambers or solid state detectors, that are located between the patient and the imaging device. During the x-ray exposure, the sensors monitor the radiation transmitted through the patient towards the imaging device and the AEC terminates the exposure when the total amount of radiation measured reaches a predetermined threshold level that corresponds for example to the required film optical density in a chosen region of the film. AEC devices assist the radiographer in producing radiographic images consistent from patient to patient regardless the patient size or presence of pathologies.

Certain AEC devices in common use are provided with several sensors and the imaging system can be set to use particular sensors of the device depending on the body part to be imaged. For example, a central AEC sensor is commonly used for spine imaging whereas two side sensors are used for lungs imaging. Thus, the term “sensor(s)” is used all through this document so as to indicate referral to one or more sensors.

Patient positioning is an important step in order to achieve the required good image quality since if the organ to be imaged is incorrectly positioned and not directly over the sensor, the radiograph will be over or underexposed. For example, in the case of film being used, the film will be either too dark or too bright. The AEC is becoming worthless equipment if the patient is poorly positioned in relation to the AEC sensors.

Patient positioning is an issue that has been increasingly dealt with over the years. Many patents deal with markers that mark the center of the x-ray beam in order to assist in patient positioning. An example is disclosed in U.S. Pat. No. 5,835,562 “Medical radiological apparatus including optical crosshair device for patient positioning and forearm and spinal positioning aides”, filed in 1995 by Ramsdell et al. This patent teaches an optical crosshair device that helps align the patient's spine along the Y-axis of the apparatus and the patient's hips along the X-axis of the apparatus, perpendicular to the spine. Another example is disclosed in U.S. Pat. No. 4,426,726 “Apparatus and method for x-ray beam alignment” filed by Cheetham in 1982. The inventor discloses an apparatus and method for aligning the center of an X-ray beam with both a reference point on the patient and the center of the x-ray film holder. Other devices that indicate the isocenter of the beam are available.

In order to effectively use the AEC system, the location of the sensor is to be indicated for the purpose of patient positioning. A locator for the AEC system is taught in U.S. Pat. No. 6,327,336 “Radiogram showing location of automatic exposure control system” by Gingold et al. According to the method disclosed in the patent, the location of the AEC sensors is displayed a posteriori, i.e. after the completion of the radiogram, on the radiogram along with the image of the subject so as to allow the observers to determine whether the radiogram image was taken with appropriate subject alignment. The main drawback of this method is the lack of any possibility to correct the patient's positioning before the image is actually taken.

Whenever possible, and as an example with wall Buckies using ionization chambers as AEC sensors, the position and size of the ionization chambers are graphically indicated on the Bucky cover. In other commonly encountered situations such as with Buckies mounted in radiographic tables with floating top or with variable Bucky position, this is not possible as the Bucky and AEC position relative to the tabletop is not fixed. Even with wall Buckies, the patient being positioned between the x-ray tube and the Bucky, obstructs the view of the AEC position indicators.

It is a long felt need to provide a simple and effective means to allow accurate patient positioning in regard with the AEC system's sensors without having to expose the patient to unnecessary radiation.

### SUMMARY OF THE INVENTION

It is an object of the present invention to provide means assisting in patient positioning for x-ray imaging that indicates the location and size of the automatic exposure control (AEC) system's sensors in relation to the patient's body and organs, all this before the exposure is being made.

It is another object of the present invention to provide an adjustable means assisting in patient positioning for x-ray imaging that can be adjusted according to the distance between the x-ray tube focal spot and the imaging device and the AEC system's sensors.

It is another object of the present invention to provide an adjustable means assisting in patient positioning for x-ray imaging that can be adjusted according to the organ or body part being imaged, the imaging procedure and the corresponding active sensor(s) of the AEC device.

It is thus provided in accordance with the present invention, a means for improving patient positioning in relation to automatic exposure control (AEC) sensor(s) before imaging takes place in an x-ray imaging system provided with an x-ray tube, collimator provided with a light source, AEC system's sensors and an imaging device, the means for improving patient positioning comprising means adapted to indicate position and field size covered by the AEC system's sensors onto the patient.

Furthermore, in accordance with another preferred embodiment the present invention comprises a light source associated with said means adapted to indicate position and field size.

Furthermore, in accordance with another preferred embodiment of the present invention, said means is a plate associated with the collimator, wherein said plate is provided with contour lines drawn onto it, whereby when light from the light source is projected through said plate, said contour lines are projected onto the patient to substantially the actual size and location of the AEC system's sensors so as to allow accurate positioning of the patient relative to the AEC system's sensors and acquiring high quality x-ray images.

Furthermore, in accordance with another preferred embodiment of the present invention, said plate is incorporated adjacent the collimator so as to allow light emitted of the light source to pass through said plate.

Furthermore, in accordance with another preferred embodiment of the present invention, the collimator is provided with auxiliary rails adapted to receive said plate.

Furthermore, in accordance with another preferred embodiment of the present invention, said plate is incorporated within the collimator so as to allow the light emitted of the light source to pass through said plate.

Furthermore, in accordance with another preferred embodiment of the present invention, said plate is a transparent plate.

Furthermore, in accordance with another preferred embodiment of the present invention, said plate is made of an acrylic material.

Furthermore, in accordance with another preferred embodiment of the present invention, said contour lines are drawn with a light opaque paint.

Furthermore, in accordance with another preferred embodiment of the present invention, said contour lines represent one or more AEC system's sensor(s) and are adjusted so as to accord a predetermined source to image distance, SID.

Furthermore, in accordance with another preferred embodiment of the present invention, said contour lines represent one or more AEC system's sensor(s) and are adjusted so as to accord more than one SID.

Furthermore, in accordance with another preferred embodiment of the present invention, at least one AEC system's sensor corresponding to a specific body part or organ, and/or a specific imaging procedure is adapted to be represented by said contour lines that are adjusted to accord the specific body part or organ being imaged and/or the specific imaging procedure and one or more than one SID.

Furthermore, in accordance with another preferred embodiment of the present invention, said means adapted to indicate position and field size comprises a set of plates associated with the collimator, wherein each plate is provided with predetermined contour lines drawn onto it, whereby when light from the light source is projected through the plate, said contour lines are projected onto the patient to substantially the actual size and location of the AEC system's sensor(s) so as to allow accurate positioning of the patient relative to the sensor(s) and acquiring high quality x-ray images.

Furthermore, in accordance with another preferred embodiment of the present invention, each plate of said set of plates is an adjustable plate having contour lines adjustable to a corresponding SID and/or a specific body part or organ and/or a specific imaging procedure.

Furthermore, in accordance with another preferred embodiment of the present invention, said set of plates is housed in a housing.

Furthermore, in accordance with another preferred embodiment of the present invention, said set of plates is incorporated adjacent the collimator and each plate of said set of plates can be positioned beneath the collimator so as to allow light emitted of the light source to pass through the plate.

Furthermore, in accordance with another preferred embodiment of the present invention, the collimator is provided with auxiliary rails adapted to receive the plate chosen from the set of plates.

Furthermore, in accordance with another preferred embodiment of the present invention, said set of plates is incorporated within the collimator so as to allow light emitted of the light source to pass through the plate chosen from the set of plates.

Furthermore, in accordance with another preferred embodiment of the present invention, said means adapted to indicate position and field size further comprises a mechanism allowing insertion of a chosen plate of said set of plates into a light beam emitted of the light source.

Furthermore, in accordance with another preferred embodiment of the present invention, said mechanism is actuated by the X-ray imaging system, based on the specific body part or organ being imaged and/or a specific imaging procedure and the SID.

Furthermore, in accordance with another preferred embodiment of the present invention, said means is an electronic means incorporated in the collimator and used to generate an opaque pattern, adjusted so as to accord the actual SID and all or part of the AEC system's sensors as required by the organ and/or imaging procedure, to be projected through the collimator onto the patient to substantially the actual size and location of the AEC system's sensor(s) so as to allow accurate positioning of the patient and acquiring high quality x-ray images.

Furthermore, in accordance with another preferred embodiment of the present invention, the X-ray imaging system feeds coordinates used to generate said opaque pattern, wherein said coordinates are based on actual SID and AEC system's sensors to be used in a given imaging procedure.

Furthermore, in accordance with another preferred embodiment of the present invention, said electronic means is mounted outside the collimator and uses its own light source.

Furthermore, in accordance with another preferred embodiment of the present invention, said means further comprises a laser and laser scanner adapted to generate, under the control of the X-ray imaging system, a pattern within the collimator and whereby said pattern is adapted to be projected through the collimator onto the patient to substantially the actual size and location of the AEC system's sensor(s) so as to allow accurate positioning of the patient and acquiring high quality x-ray images.

Furthermore, in accordance with another preferred embodiment of the present invention, said pattern accords a predetermined SID.

Furthermore, in accordance with another preferred embodiment of the present invention, said pattern accords specific AEC system's sensor(s) needed for an imaged organ and/or imaging procedure.

Furthermore, in accordance with another preferred embodiment of the present invention, said laser and laser scanner generate said pattern on a mirror system provided within the collimator and wherein said mirror system is adapted to transfer said pattern onto the patient.

Furthermore, in accordance with another preferred embodiment of the present invention, said laser and said laser scanner together with said mirror system are mounted in an enclosure outside the collimator and are adjusted so as to match the light field of the collimator.

Furthermore, in accordance with another preferred embodiment of the present invention, said pattern is projected onto the patient while the light source is turned off.

Furthermore, in accordance with another preferred embodiment of the present invention, the imaging device can be selected from a group of film screen, radiography flat panel detector, real time flat panel detector as used for fluoroscopy or radiography, fluorescent or scintillating screen coupled to image intensifier, fluorescent or scintillating screen coupled to a CCD or CMOS camera, or any other X-ray imaging detector.

In accordance with yet another preferred embodiment of the present invention, it is further provided a method for improving patient positioning relative to AEC system's sensor(s) before x-ray imaging, said method comprising:
providing a means to indicate onto the patient actual location and size of fields covered by the AEC system's sensor(s);
projecting light through said means so as to indicate said fields;
positioning the patient according to said fields.

Furthermore, in accordance with another preferred embodiment of the present invention, said means is a plate provided with contour lines drawn onto it and wherein said contour lines indicate said fields.

Furthermore, in accordance with another preferred embodiment of the present invention, said means is a set of plates provided with contour lines drawn on to them and wherein said contour lines indicate said fields.

Furthermore, in accordance with another preferred embodiment of the present invention, said means comprises an electronic means adapted to provide a pattern to be projected on the patient wherein said pattern is adapted to indicate said fields.

Furthermore, in accordance with another preferred embodiment of the present invention, said means comprises a laser and laser scanner adapted to generate a pattern corresponding to said fields.

### BRIEF DESCRIPTION OF THE FIGURES

In order to better understand the present invention and appreciate its practical applications, the following Figures are attached and referenced herein. Like components are denoted by like reference numerals.

It should be noted that the figures are given as examples and preferred embodiments only and in no way limit the scope of the present invention as defined in the appending Description and Claims.

FIG. 1a illustrates an isometric view of a device for indication of the AEC system's sensors onto a patient before exposure to radiation in accordance with a preferred embodiment of the present invention.

FIG. 1b illustrates a frontal view of the device shown in FIG. 1a.

FIG. 2 schematically illustrates a device for indication of the AEC system's sensors onto a patient in accordance with a preferred embodiment of the present invention, incorporated in an X-Ray imaging system.

FIG. 3 illustrates a contour of the marker of an AEC sensor on the device in accordance with a preferred embodiment of the present invention and its projections on the patient and the imaging device.

FIG. 4 illustrates a projection of markers made by the device in accordance with a preferred embodiment of the present invention indicating the position of an AEC system's sensors and their size on a patient lying in an imaging system before exposure to x-ray beam.

FIG. 5 schematically illustrates a device for indication of the AEC system's sensors onto a patient in accordance with another preferred embodiment of the present invention, incorporated in an X-Ray imaging system.

FIG. 6 illustrates a schematic cross sectional view of a collimator system incorporated with means for positioning a patient in accordance with another preferred embodiment of the present invention.

FIG. 7 schematically illustrates a device for generating an electronically controlled adjustable opaque pattern in accordance with another preferred embodiment of the present invention.

FIG. 8 illustrates a schematic cross sectional view of a collimator system incorporated with an adjustable pattern generator based on a laser and a scanner in accordance with a preferred embodiment of the present invention.

### DETAILED DESCRIPTION OF THE INVENTION AND FIGURES

The present invention provides unique and novel devices facilitating proper positioning of a patient in an X-Ray imaging system. The means for improving the positioning of a patient according to the present invention enables an effective exploitation and use of the AEC system that is typically positioned beneath or behind the patient.

Although an organ to be imaged should be positioned relative to the placement of the AEC system's sensor(s), the majority of the positioning aids that are available in the art provide an indication of the X-Ray beam's center and/or dimensions but none provides positioning indication of the AEC system's sensor(s). The present invention provides effective means allowing proper positioning of the patient relative to the AEC system's sensor(s) before he is exposed to the beam. The positioning of the patient relative to the AEC system's sensor(s) can be corrected before any exposure occurs, thus both improving image quality and saving the patient unnecessary radiation exposure.

In accordance with a method in which the positioning of a patient in an X-Ray imaging system is improved by the means and methods subject of the present invention, the position and size of the field covered by the AEC sensors are projected on the patient's body before imaging occurs. These projections are at the same location and cover substantially the same patient area as covered by the actual AEC system's sensors. The patient is positioned according to the AEC sensor's pattern projected on his body or organ so as to acquire an image having better quality than the quality of images taken on the basis of the radiographer's experience and intuition.

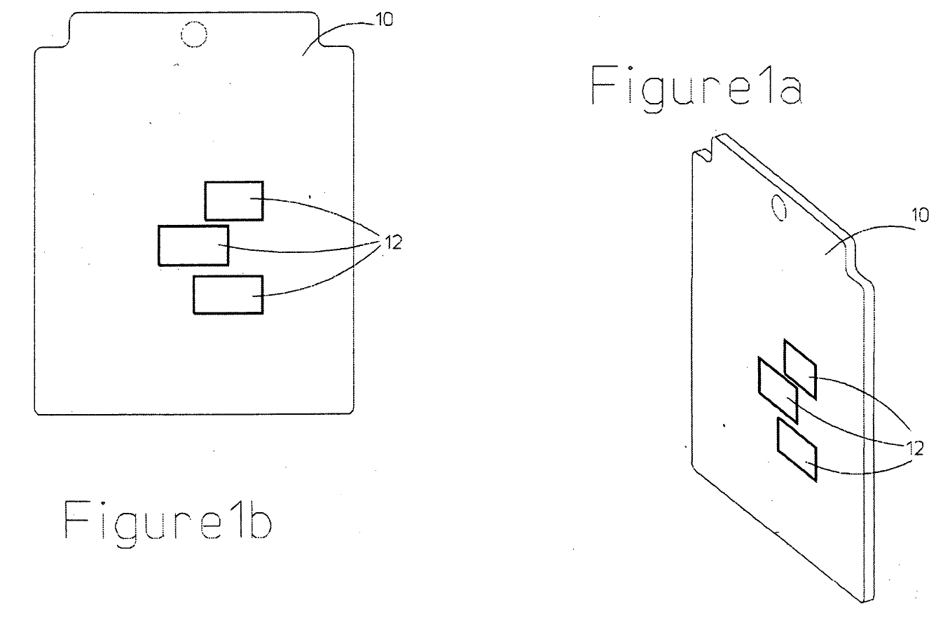

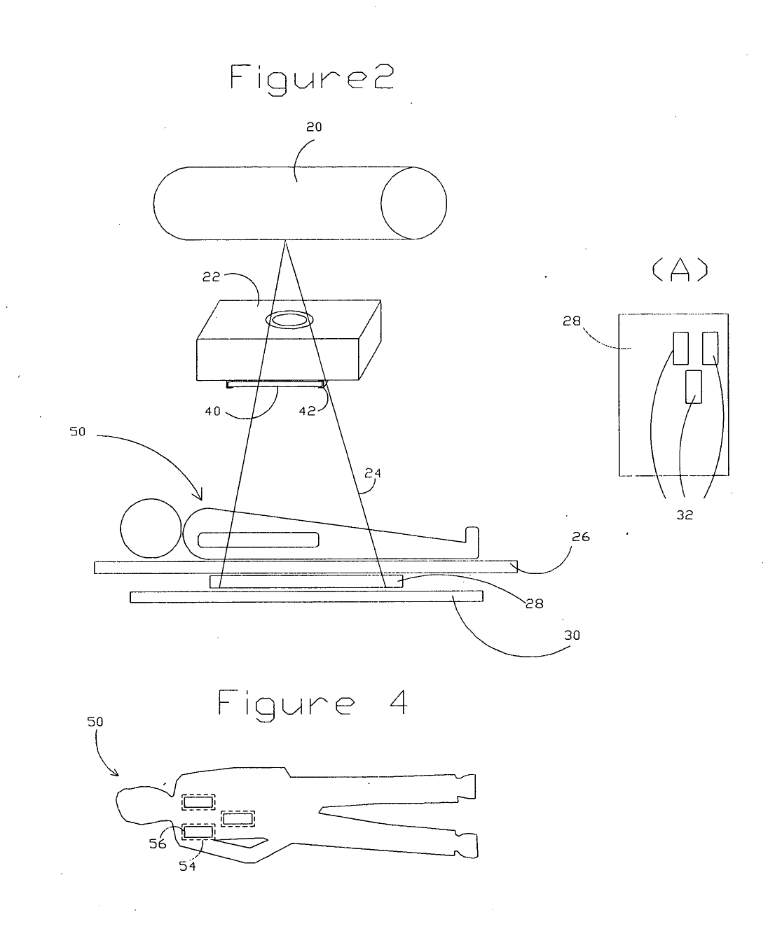

Reference is now made to FIGS. 1a and 1b illustrating an isometric view and a front view of a device for indication of the AEC system's sensors onto a patient before exposure to radiation in accordance with a preferred embodiment of the present invention. A plate 10 is a transparent plate made for example of acrylic material on which contour lines 12 are drawn. Contour lines 12 mark the properly scaled circumference of the AEC's sensors that are provided in a specific X-Ray imaging system. The contour is located on the device and sized to an extent of which when the contour is projected onto the patient, it substantially represents the actual location and size of the AEC's sensors that are provided in the imaging system in front of the imaging device. Contour lines 12 are preferably drawn with a light opaque paint that results in a darker pattern when projected onto the patient.

Plate 10 may be used to project the pattern of all AEC sensors on the patient, in which case the radiographer shall decide which of the sensors is relevant for a particular body part. Alternatively, different plates 10 may be provided for different body parts and/or different imaging procedures, each projecting the pattern of one or more particular AEC sensors.

Reference is now made to FIG. 2 schematically illustrating a device for indication of the AEC system's sensors onto a patient in accordance with a preferred embodiment of the present invention, incorporated in an X-Ray imaging system. The device of the present invention can be incorporated in an existing x-ray imaging system having an x-ray tube 20 provided with a collimator 22 that directs an X-ray beam indicated by lines 24 onto table 26 beneath which AEC system's sensors 28 are provided. Adjacently beneath AEC system's sensors 28, an imaging device 30 is provided. The imaging device type depends on the specific system used and can be a film screen, or a radiography flat panel detector, or a real time flat panel detector as used for fluoroscopy or radiography, or a fluorescent or scintillating screen coupled to image intensifier, or a fluorescent or scintillating screen coupled to a CCD or CMOS camera, or any other technology available. An example of such AEC system's sensors used for radiography is shown in a frontal view, for clarity, in image [A], and has three ionization chambers mounted in a pattern 32. It should be mentioned that the number, shape, size and location of the sensors could change from system to system.

A patient 50 to be imaged is lying on table 26 wherein the patient has to be positioned by the radiographer with the organ in question on top of pattern 32. This pattern is not drawn on table 26 since the patient that lies on the table would hide it. Also with floating top tables, the tabletop on which the patient lies has not a fixed position relative to the AEC system's sensors 28 and the imaging device 30. However, the quality of the resulting image depends on the position of the organ in question relative to the pattern 32.

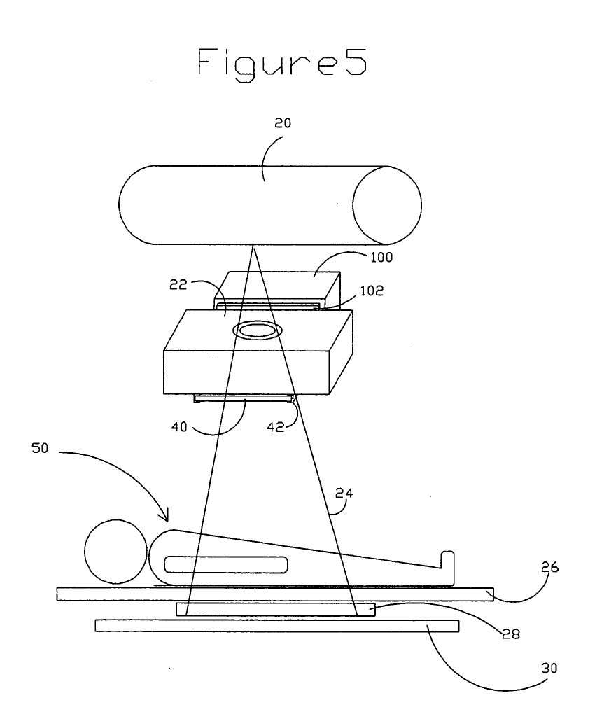

According to one aspect of the present invention, a device for improved patient positioning 40 similar to device 10 shown in FIG. 1 is inserted within auxiliary rails 42 that are usually available with x-ray collimator 22. Auxiliary rails 42 are mounted on the beam outlet and usually serve for the insertion of other aids such as filters, calibration plates, etc. The collimator is provided with a light source that generates a light beam using a mirror system (the light source and mirror system are not shown in FIG. 2). The light beam is collimated to substantially the same extent as the X-Ray beam 24. When the light source is turned on, light shines through the device for improved positioning 40, the contour that is drawn on the device is projected onto the patient marking the pattern of the AEC system's sensors, as shown in FIG. 4.

Moreover, according to another aspect of the present invention, a set of plates is provided so as to allow versatility of contours. Each plate is adjusted to a particular AEC sensor, or group of sensors (for example, one of the sensors indicated on FIG. 2A) as required for a certain body part and/or imaging procedure. Alternatively, each plate will carry the contour(s) of the AEC sensor(s) adjusted to a specific Source to Image Distance (SID) as shown herein after.

The particular relevant plate can be selected from the given set by the radiographer and inserted manually into rails 42. Alternatively, this can be done with a mechanical or electromechanical actuator according to a command made by the radiographer and delivered to the system. Furthermore, as with more integrated and automated systems, the command for the insertion of a particular plate is issued by the radiography system according to the selected imaging procedure being performed and eventually also the SID measured by the system.

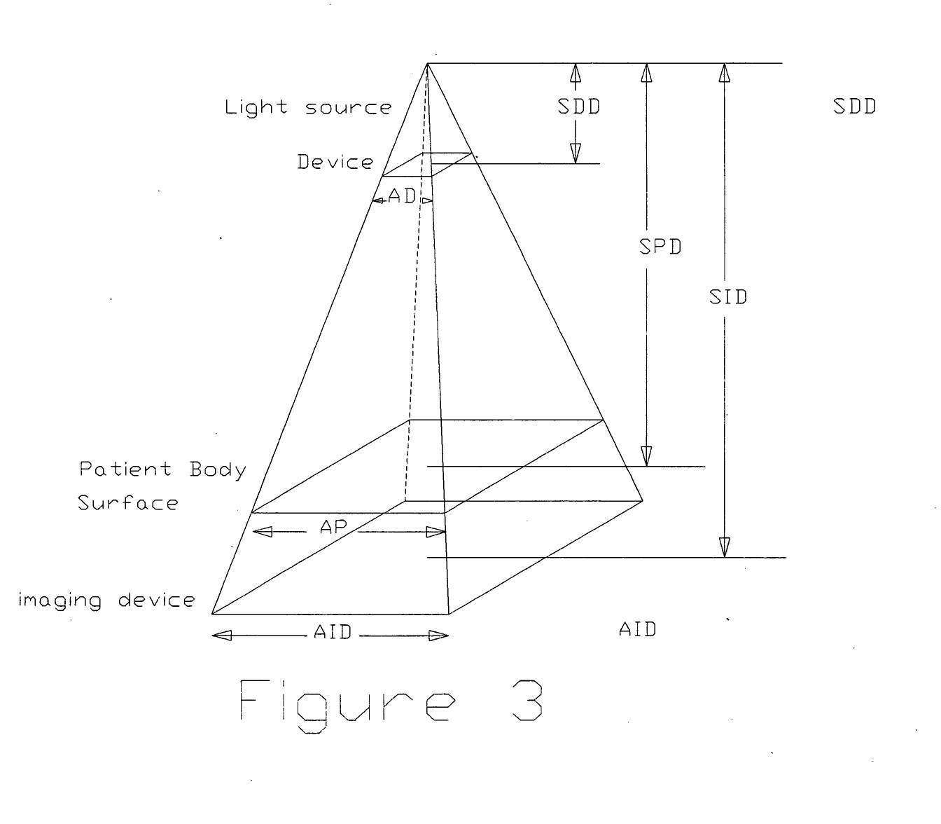

Reference is now made to FIG. 3 illustrating the contour of a device in accordance with a preferred embodiment of the present invention and its projections on the patient body surface and the imaging device. The above-mentioned indication that the light and X-Ray beams are collimated to substantially the same extent results in a light source to image distance (SID) being substantially identical to the X-ray SID. The contour sizes are AD, which is the dimension of the AEC sensor in the device's plane, AP, which is the dimension of the AEC sensor projected on the patient's body surface and AID, which is the dimension of the AEC sensor in the imaging device plane. As shown below, AID is practically also the actual AEC sensor's size. The relationship between the above dimensions are dictated by the AID and the SDD, SPD and SID, which are the source to device distance, source to patient distance and source to image distance, respectively. For all practical purposes, the gap between the AEC sensors and the imaging device is very small and negligible, and therefore:
AD/AID=SDD/SID (1)

This relationship indicates that one has to use different contours (AD) according to the SID.

Wall Buckies that are operated normally at either 1 m SID or at 1.8 m SID can be provided with two different devices, each for a given SID. Optionally, the device for improving patient positioning can be provided with two drawn contours, each adapted to project a size that will correctly represent the pattern of the AEC ionization chambers for a given SID. It is optional to draw each contour with different opaque paint so as to ease the radiographer to distinguish between them.

As seen from FIG. 3, AP<AID, i.e. the projected size on the patient's body surface is somewhat smaller (by about 20%-30% for SID 1 m and about 10%-15% for SID 1.8 m) than the actual size of the AEC sensor. This is a small and acceptable error for all practical purposes. Furthermore, this should not disturb in optimal and correct patient and organ positioning above the AEC sensor since the area indicated on the patient properly represents the spatial angle covered by the AEC sensors in relation to the X-ray beam source.

The discrepancy between AP and AID is due to the thickness of the patient's body, or body part being imaged and the specific geometry of the given imaging system (i.e. distance between tabletop and imaging device). One could diminish this discrepancy by increasing AD beyond the value resulting from equation (1), based on a typical body thickness value and adapted to the specific imaging system. This will however still leave some inaccuracies due to patient-to-patient variability.

Reference is now made to FIG. 4 illustrating a projection of markers made by the device of the present invention indicating the positioning of an AEC system's sensors on a patient lying in an X-Ray imaging system before exposure to x-ray beam. A patient 50 is preferably lying on a table; however, the patient can also stand if a wall Bucky is used. Before imaging the patient by the x-ray beam, light is projected onto the patient through the collimator as shown before and through the device for improving the patient positioning. Contour 12 that is drawn on device 10 (as shown in FIG. 1 and corresponding to AD in FIG. 3) is projected as contour 56 (corresponding to to AP in FIG. 3) on patient 50, while the actual size of the AEC system's sensors is indicated by contour 54 (corresponding to AID in FIG. 3). As already explained before, the projected contour 56 on the patient (depicted by solid line in FIG. 4) is slightly smaller than the actual size of the AEC sensor 54 (dotted line).

The illustration shows a case in which the shoulder of patient 50 is to be imaged. The radiographer is positioning the patient in a way in which the shoulder is exactly on top of the contour of an ionization chamber 54 that will be the one activating the AEC. Miss-positioning the patient so that ionization chamber 54 is, for example, partially beneath the shoulder and partially beneath vacancy may result in an image having poor quality.

Reference is now made to FIG. 5 illustrating a device for indication of the AEC system's sensors onto a patient in accordance with another preferred embodiment of the present invention, incorporated in an X-Ray imaging system. The X-ray imaging system is similar to the embodiment shown in FIG. 2, however, a housing 100 is provided adjacent to x-ray collimator 22. Housing 100 is provided with a set of plates 102 positioned in a way by which each one of the plates can be directed to be inserted beneath x-ray collimator 22 and within auxiliary rails 42. The choice and direction of the appropriate plate to beneath the collimator is preferably performed automatically by the imaging system (based on the measured SID, and/or the imaged organ and/or the imaging procedure), or as indicated by the radiographer, as shown before. It should be noted that any actuator that directs an appropriate device for positioning a patient from a set of devices to the collimator so as to allow its projection on the patient could be utilized in any of the embodiments mentioned herein without limiting the scope of the present invention.

Optionally, the set of plates 102 comprises two or more plates as needed, each of the plates is provided with the appropriate contour that is adjusted to the specific SID. Alternatively, set 102 will comprise more plates according to the specific organ and/or imaging procedure. For example, if one images the spine, the central sensor (as shown in FIG. 2A) will be needed and a device on which only this contour is marked will be chosen. In the case of lung imaging, the two lateral sensors are needed and the chosen device will have these two contours marked.

The specific device is chosen from the set of devices, either as a device with the contour of all AEC sensors or according to the specific organ and/or imaging procedure performed and/or the correct SID (as sensed by the imaging system or indicated by the radiographer). This chosen device is inserted into light beam 24, thus projecting the AEC sensor's pattern on the patient. It will carry the opaque pattern, properly sized and ensuring the correct projected size of the AEC sensors on the patient, as described before.

As mentioned herein before, the devices for improving the positioning of patient can be provided and mounted externally to the collimator system, as illustrated in FIG. 5.

In another aspect of the present invention, the device for improved patient positioning is provided and incorporated within the collimator. In some cases, the device for improved patient positioning is a part of a set of devices mounted in the collimator.

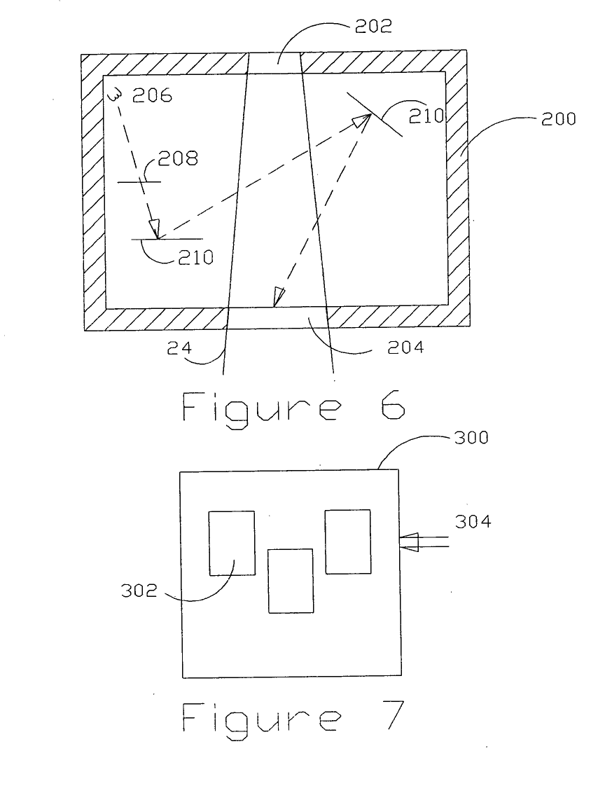

Reference is now made to FIG. 6 schematically illustrating a cross sectional view of a collimator system incorporated with means for positioning a patient. Collimator system 200 is provided with an upper opening 202 through which the x-ray beam enters from an X-Ray tube and a lower opening 204 through which a collimated x-ray beam 24 exits the collimator and is transferred to a patient as well as a light beam of the same collimation characteristics, that is generated by a light source 206 and a system of mirrors 210 (schematically drawn). Means for positioning a patient 208 is placed in the light beam between light source 206 and mirror device 210 that directs the light beam to the lower opening 204. Means for positioning a patient 208 can be a single plate or a plate selected from a set of plates carrying the contour of all AEC sensors, or only one or more specific sensors, for one SID or for more SIDs. The specific plate to be used is chosen in accordance with the adjustments made to the x-ray system automatically or by the radiographer, as described herein before. The device will carry the opaque pattern, properly sized and ensuring the correct projected size of the AEC sensors on the patient, as described before. The specific plate to be used will be inserted into the light beam, at the position of plate 208, by a mechanism similar to the one described herein above.

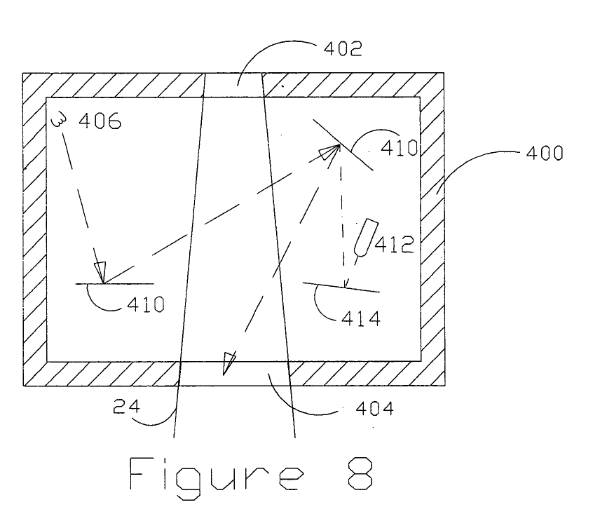

According to another aspect of the present invention, the device for improving patient positioning, as needed in all above mentioned embodiments, is an adjustable device that can generate an opaque pattern adjusted so as to accord the actual SID and all AEC sensors or only the specific AEC sensor(s) as required by the imaged organ and/or imaging procedure being performed. In a preferred embodiment, illustrated schematically in FIG. 7, a device 300 capable of generating an electronically controlled adjustable opaque pattern and preferably a light valve (not shown in the figure) is used so as to allow an opaque pattern 302 to adapt to an actual SID and/or specific AEC sensor(s) as required by the organ and/or imaging procedure. The light valve receives the coordinates of required opaque pattern 302 from the imaging system by command 304 and generates this opaque pattern that will appear on the device, properly scaled and according to the size and location of the AEC sensors. Other than light valve technologies for generating the opaque pattern in a controlled manner can also be used.

The method by which the adjustable device for improving patient positioning before x-ray imaging is used is as follows: a device 300 provided with electronically generated contour lines is located either adjacently to the collimator (e.g. mounted at the X-Ray and light beam outlet from the collimator, in a position similar to plate 40 in FIG. 2) or integrated in the collimator, similar to device 208 shown in FIG. 6. Alternatively, the adjustable device can be mounted in a separate enclosure, provided with its own light source, in which case this source has to be adjusted in order to match the light field of the collimator. The X-ray imaging system measures the SID and calculates the coordinates for the contour(s) 302 of the opaque pattern on the plate (based on SID and/or imaged organ and/or imaging procedure and adequate scaling). These coordinates are issued to the plate (preferably light valve) on which the adjusted opaque contour(s) are generated. Then, light is projected through the plate and adjusted opaque contours 302 are projected onto the patient. Positioning of the patient can now be done according to AEC sensor(s) contour(s) adapted to the actual SID and/or organ and/or imaging procedure being performed.

In yet another aspect of the present invention, using a laser and a scanner mounted preferably inside the collimator generates the AEC sensor pattern, adequately sized.

Reference is now made to FIG. 8 illustrating a schematic cross sectional view of a collimator incorporated with an adjustable pattern generator in accordance with a preferred embodiment of the present invention. The pattern generated by a laser-scanner combination is projected onto the collimator mirror and from there on to the patient. For that reason, collimator 400 that is provided with an upper opening 402 and an lower opening 404 that are adapted to allow the x-ray to pass onto the patient is equipped with a light source 406 and a mirror system 410 adapted to direct the light to pass through lower opening 404 and onto the patient. A laser 412 and scanner 414 are provided and are adapted to project an appropriate pattern (matched to the SID and/or organ and/or imaging procedure, under the control of the imaging system) onto mirror system 410 and from there onto the patient. The pattern projected on the patient will be similar to that shown in FIG. 4. The collimator light may be turned off, thus a light pattern (instead of the shadow given by the opaque pattern described herein before) representing the AEC sensor pattern is projected, when desired, on the patient. In this embodiment, the patient is first positioned according to the radiographer's knowledge and using the collimator's light field. Then the final patient positioning is performed after having turned the collimator light source 406 off and laser-scanner 412 and 414 on. However, turning the collimator light source off is optional. One may leave the light on, in which case the laser generated AEC sensor pattern will project on the patient in a fashion similar with the projection of a laser generated cross hair (indicating the central axes of the X-Ray field) in the light field.

In another embodiment, laser 412 and scanner 414 may be mounted externally to the collimator in a box adjacent to the collimator, similar to box 100 shown on FIG. 5. In this case, an additional mirror system is needed for projecting the pattern on the patient. The laser light pattern generated by the laser-scanner under the control of the imaging system will project onto the patient as shown previously, i.e. either alone (with the collimator light turned off) or superimposed on the light beam emerging from the collimator. It is mandatory that the above-described device matches the collimator's light field.

It should be clear that the description of the embodiments and attached Figures set forth in this specification serves only for a better understanding of the invention, without limiting its scope as covered by the following claims. In particular, other techniques to generate projected light patterns may be used and are covered in the scope of this invention.

It should also be clear that a person skilled in the art, after reading the present specification can make adjustments or amendments to the attached Figures and above described embodiments that would still be covered by the following claims.

1. A means for improving patient positioning in relation to automatic exposure control (AEC) system's sensor(s) before imaging takes place in an x-ray imaging system provided with an x-ray tube, collimator provided with a first light source, AEC system's sensor(s) and an imaging device, the means for improving patient positioning comprising means adapted to indicate position and field size covered by the AEC system's sensor(s) onto the patient.

2. The means as claimed in claim 1, further comprises a second light source independent of the collimator's first light source and associated with said means adapted to indicate position and field size.

3. The means as claimed in claim 1 wherein said means is a plate associated with the collimator, wherein said plate is provided with contour lines drawn onto it, whereby when light from the first light source is projected through said plate, said contour lines are projected onto the patient to substantially the actual size and location of the AEC system's sensor(s) so as to allow accurate positioning of the patient relative to the AEC system's sensor(s) and acquiring high quality x-ray images.

4. The means as claimed in claim 3, wherein said plate is incorporated adjacent the collimator so as to allow light emitted of the first light source to pass through said plate.

5. The means as claimed in claim 4, wherein the collimator is provided with auxiliary rails adapted to receive said plate.

6. The means as claimed in claim 3, wherein said plate is incorporated within the collimator so as to allow the light emitted of the first light source to pass through said plate.

7. The means as claimed in claim 3, wherein said plate is a transparent plate.

8. The means as claimed in claim 3, wherein said plate is made of an acrylic material.

9. The means as claimed in claim 3, wherein said contour lines are drawn with a light opaque paint.

10. The means as claimed in claim 3, wherein said contour lines represent one or more of the AEC system's sensors and are adjusted so as to accord a predetermined source to image distance, SID.

11. The means as claimed in claim 3, wherein said contour lines represent one or more of the AEC system's sensors and are adjusted so as to accord more than one SID.

12. The means as claimed in claim 3, wherein at least one AEC system's sensor corresponding to a specific body part or organ, and/or a specific imaging procedure is adapted to be represented by said contour lines that are adjusted to accord the specific body part or organ being imaged and/or the specific imaging procedure and one or more than one SID.

13. The means as claimed in claim 2 wherein said means is a plate associated with the collimator, wherein said plate is provided with contour lines drawn onto it, whereby when light from said second light source is projected through said plate, said contour lines are projected onto the patient to substantially the actual size and location of the AEC system's sensor(s) so as to allow accurate positioning of the patient relative to the AEC system's sensor(s) and acquiring high quality x-ray images.

14. The means as claimed in claim 13, wherein the collimator is provided with auxiliary rails adapted to receive said plate.

15. The means as claimed in claim 13, wherein said plate is incorporated within the collimator so as to allow the light emitted of the first light source to pass through said plate.

16. The means as claimed in claim 13, wherein said plate is a transparent plate.

17. The means as claimed in claim 13, wherein said plate is made of an acrylic material.

18. The means as claimed in claim 13, wherein said contour lines are drawn with a light opaque paint.

19. The means as claimed in claim 13, wherein said contour lines represent one or more of the AEC system's sensors and are adjusted so as to accord a predetermined source to image distance, SID.

20. The means as claimed in claim 13, wherein said contour lines represent one or more of the AEC system's sensors and are adjusted so as to accord more than one SID.

21. The means as claimed in claim 13, wherein at least one AEC system's sensor corresponding to a specific body part or organ, and/or a specific imaging procedure is adapted to be represented by said contour lines that are adjusted to accord the specific body part or organ being imaged and/or the specific imaging procedure and one or more than one SID.

22. The means as claimed in claim 1, wherein said means adapted to indicate position and field size comprises a set of plates associated with the collimator, wherein each plate is provided with predetermined contour lines drawn onto it, whereby when light is projected through the plate, said contour lines are projected onto the patient to substantially the actual size and location of the AEC system's sensor(s) so as to allow accurate positioning of the patient relative to the sensor(s) and acquiring high quality x-ray images.

23. The means as claimed in claim 22, wherein each plate of said set of plates is an adjustable plate having contour lines corresponding to parameters such as SID and/or a specific body part or organ, and/or a specific imaging procedure.

24. The means as claimed in claim 22, wherein said set of plates is housed in a housing.

25. The means as claimed in claim 22, wherein said set of plates is incorporated adjacent to the collimator and each plate of said set of plates can be positioned beneath the collimator so as to allow said light to pass through the plate.

26. The means as claimed in claim 22, wherein the collimator is provided with auxiliary rails adapted to receive the plate chosen from the set of plates.

27. The means as claimed in claim 22, wherein said set of plates is incorporated within the collimator so as to allow said light to pass through the plate chosen from the set of plates.

28. The means as claimed in claim 22, wherein said predetermined contour lines represent a corresponding SID and/or a specific body part or organ, and/or a specific imaging procedure.

29. The means as claimed in claim 22, wherein said means adapted to indicate position and field size further comprises a mechanism allowing insertion of a chosen plate of said set of plates into said light's beam.

30. The means as claimed in claim 29, wherein said mechanism is actuated by the X-Ray imaging system, based on the specific body part or organ being imaged and/or a specific imaging procedure and the SID.

31. The means as claimed in claim 1, wherein said means is an electronic means incorporated in the collimator and used to generate an opaque pattern, adjusted so as to accord the actual SID and all or part of the AEC system's sensors as required by the organ and/or imaging procedure, to be projected through the collimator onto the patient to substantially the actual size and location of the AEC system's sensor(s) so as to allow accurate positioning of the patient and acquiring high quality x-ray images.

32. The means as claimed in claim 31, wherein the X-ray imaging system feeds coordinates used to generate said opaque pattern, wherein said coordinates are based on actual SID and AEC system's sensor(s) to be used in a given imaging procedure.

33. The means as claimed in claim 2, wherein the X-ray imaging system feeds coordinates used to generate said opaque pattern, wherein said coordinates are based on actual SID and AEC system's sensor(s) to be used in a given imaging procedure.

34. The means as claimed in claim 31, wherein said electronic means is mounted outside the collimator and uses an independent third light source.

35. The means as claimed in claim 1, further comprises a laser and laser scanner adapted to generate, under the control of the X-ray imaging system, a pattern within the collimator and whereby said pattern is adapted to be projected through the collimator onto the patient to substantially the actual size and location of the AEC system's sensors so as to allow accurate positioning of the patient and acquiring high quality x-ray images.

36. The means as claimed in claim 35, wherein said pattern accords a predetermined SID.

37. The means as claimed in claim 35, wherein said pattern accords specific AEC system's sensor(s) needed for an imaged organ and/or imaging procedure.

38. The means as claimed in claim 35, wherein said laser and laser scanner generate said pattern on a mirror system provided within the collimator and wherein said mirror system is adapted to transfer said pattern onto the patient.

39. The means as claimed in claims 38, wherein said laser and said laser scanner together with said mirror system are mounted in an enclosure outside the collimator and are adjusted so as to match the light field of the collimator.

40. The means as claimed in claims 39, wherein said pattern is projected onto the patient while the first light source is turned off.

41. The means as claimed in claim 1, wherein the imaging device can be selected from a group comprising film screen, radiography flat panel detector, real time flat panel detector as used for fluoroscopy or radiography, fluorescent or scintillating screen coupled to image intensifier, fluorescent or scintillating screen coupled to a CCD or CMOS camera, or any other X-ray imaging detector.

42. The means as claimed in claim 2, wherein the imaging device can be selected from a group comprising film screen, radiography flat panel detector, real time flat panel detector as used for fluoroscopy or radiography, fluorescent or scintillating screen coupled to image intensifier, fluorescent or scintillating screen coupled to a CCD or CMOS camera, or any other X-ray imaging detector.

43. A method for improving patient positioning relative to AEC system's sensor(s) before x-ray imaging, said method comprising:
providing a means to indicate onto the patient actual location and size of fields covered by the AEC system's sensors;
projecting light through said means so as to indicate said fields;
positioning the patient according to said fields.

44. The method as claimed in claim 43, wherein said means is a plate provided with contour lines drawn onto it and wherein said contour lines indicate said fields.

45. The method as claimed in claim 43, wherein said means is a set of plates, provided with drawn contour lines and wherein said contour lines indicate said fields.

46. The method as claimed in claim 43, wherein said means comprises an electronic means adapted to provide a pattern to be projected on the patient wherein said pattern is adapted to indicate said fields.

47. The method as claimed in claim 32, wherein said means comprises a laser and laser scanner adapted to generate a pattern corresponding to said fields.
